(19) United States
(12) Patent Application Publication
(10) Pub. No.: US 2018/0021053 A1
(43) Pub. Date: Jan. 25, 2018

(54) BRUSH FOR ABRADING THE PARIETAL PLEURA

(71) Applicants: Christian CHOUX, Savigneux (FR); Bernard PAIN, Monistrol Sur Loire (FR); Centre Hospitalier Universitaire, Saint-Etienne (FR); Université Jean Monnet, Saint Etienne (FR)

(72) Inventors: Christian CHOUX, Savigneux (FR); Bernard PAIN, Monistrol Sur Loire (FR); Olivier TIFFET, Saint-Just-Saint-Rambert (FR)

(21) Appl. No.: 15/547,292

(22) PCT Filed: Feb. 4, 2016
(86) PCT No.: PCT/EP2016/052414

§ 371 Date: Jul. 28, 2017

(30) Foreign Application Priority Data
Feb. 11, 2015 (FR) 1551090

(51) Int. Cl. A61B 17/32; A46D 1/00; A46B 15/00; A46B 7/02

(52) U.S. Cl. CPC A61B 17/320016; A46B 7/023; A46D 1/0207; A46B 15/0085; A61B 2017/320012; A61B 2017/00809

(57) ABSTRACT

A disposable brush which allows the pleura to be abraded using pleuroscopy and allows surgeons to adjust the stiffness of the brush and the area to be abraded during the abrasion process includes a longitudinal body which supports rigid bristles and slides within a tubular member that is secured to a handle. A manually actuated slide is connected to the longitudinal body and is movable within a longitudinal lumen of the handle and cooperates with an indexing component supported on the edges of the lumen. Furthermore, the rigid bristles have elastically deformable cambers with different radii of curvature so as to form a flat fan-shaped brush in the deployed operating position outside the tubular member. The bristles of the brush extend laterally from the longitudinal axis.

### TECHNICAL FIELD

The disclosure relates to the surgical treatment by laparoscopy of a pneumothorax being characterized by the detachment of the parietal pleura from the thorax.

### BACKGROUND

Currently, the treatment comprises an abrasion or scraping phase during which the surgeon irritates by laparoscopy the parietal pleura which adheres to the ribs with an abrasive material, so that the resulting bleeding generates, by healing, adhesions facilitating the subsequent bonding of the parietal pleura with the visceral pleura.

This technique gives satisfactory medical results but is binding on the surgeon who must cut fragments of abrasive foam to make them have a size allowing them to be grasped by a plier passing through a <<thoracoscopic>> trocar.

It is known from the document FR2981840 a pleural abrasion device comprising:

a longitudinal body carrying at one of its ends strands forming a scraping brush,

a tubular body surrounding the longitudinal body and, disposed in a handle,

means communicating with the brush and the longitudinal body carrying it, a movement of displacement relative to the tubular body.

The brush can be displaced between a storage position, in which it is retracted in the tubular body and a working position, in which it protrudes out of the free end of the tubular body, and is animated by a scraping movement, for example rotary movement.

Since these strands or bristles are made of a polyamide material, such as the one known under the trademark <<NYLON>>, the abrasion obtained is low and hardly controllable by the surgeon during operation.

In addition, the strands are oriented transversely on the side and/or longitudinally at the end, and allow carrying out the brushing only along two perpendicular directions, which is very insufficient for the surgeon working on curved intermediate portions with a tool whose orientation of the scraping means is limited and cannot be modified because of its guiding according to the longitudinal axis of the trocar.

The replacement of the strands or bristles made of polyamides by strands made of rigid material could improve the scraping power of the end of the strands, but not their stiffness which depends on their free length, that is to say a parameter which, in the current brushes, is neither adjustable nor controllable by the surgeon during the operation.

Another disadvantage results from the shape of the device which, designed for an assistance by motor means, in particular by rotation, does not allow to provide a scraping force adapted to the needs nor to modify the scraping movement as would like the surgeon.

### SUMMARY

The aim of the disclosure is to remedy these disadvantages by providing a single-use brush arranged to ensure the abrasion of the pleura by <<pleuroscopy>> without requiring preparatory operations implementing abrasion means with imperfect sterility and allowing to adapt during the operation the stiffness and the orientation of the scraping.

For this purpose, in the brush according to the disclosure, the means for displacing the longitudinal body carrying the strands are constituted by a slide which is manually actuated and secured to the posterior end of the longitudinal body, said slide being movable in a longitudinal lumen and cooperating with indexing means carried by the edges of this lumen, while the rigid material strands are provided with elastically deformable cambers having different radii of curvature so as to form, when they are retracted in the body, a bundle of parallel strands and, when they are in the working position, out of the tubular body, a flat fan-shaped brush, whose strands extend laterally from the longitudinal axis.

The brush is provided with its strands in the storage position, that is to say, completely stored in parallel and in the case formed by the end of the tubular body. This allows engaging it in the trocar of reduced diameter in order to bring it close to the area to be scraped, without these strands clinging to the vital organs of the patient, such as the lungs and the heart.

When, in the phase of introduction into the body of the patient, the surgeon decides that the action position is reached, with the aid of the indexation, it brings all or part of the strands out of the tubular body in order to form a brush having the rigidity, that is to say the <<nerve>> required and whose strands form a fan extending from the longitudinal axis of the brush to the most laterally inclined strand.

Thus, because the indexation on the handle allows visualizing the progress of the strands relative to the free end of the tubular body, the surgeon can give the strands:

the stiffness he wants, and,

a distribution corresponding to the needs of the scraping, namely more or less stiffness and a possibility of active spatial scraping at the end or laterally.

At the end of the operation, the removal of the brush is carried out rapidly, after bringing the strands back into their case, so as not to pollute the operated area.

In an embodiment of the disclosure, the handle of the brush contains inner guide rails on which the slide can move.

With this arrangement, it is the longitudinal body which is displaced relative to the tubular body forming a case. This solution increases the length of the handle, since the latter must provide for the retraction stroke of the strands, but it also has the advantage of facilitating the complete retraction of the strands inside the tubular body and the advantage of not injuring organs on its retraction path.

Advantageously, the angle a of the fan formed by the strands, measured between, on the one hand, a strand B1 parallel to the longitudinal axis of the body carrying the strands and, on the other hand, the most cambered strand B2 on the side, has a value which decreases from 180 degrees to 90 degrees as the strands move away from the strand B1 parallel to the longitudinal axis.

### BRIEF DESCRIPTION OF THE DRAWINGS

Other characteristics and advantages will become apparent from the following description, with reference to the appended schematic drawing, showing an embodiment of this plier, wherein:

FIG. 1 is a perspective view, when it is in the position of use of the coming out strands;

FIG. 2 is an exploded perspective view of the brush of FIG. 1;

FIGS. 3, 4 and 5 are side sectional views on an enlarged scale, respectively along III-III and IV-IV of FIG. 1, the strands coming out partially in FIG. 4 and coming out fully in FIG. 5;

FIG. 6 is a partial view of a variant of the strands of the brush allowing the latter to form a fan over substantially 90 degrees; and

FIGS. 7 and 8 are respectively sectional and elevation views of the brush of FIG. 6 in the working position, with the strands coming out to the maximum.

### DETAILED DESCRIPTION OF THE DRAWINGS

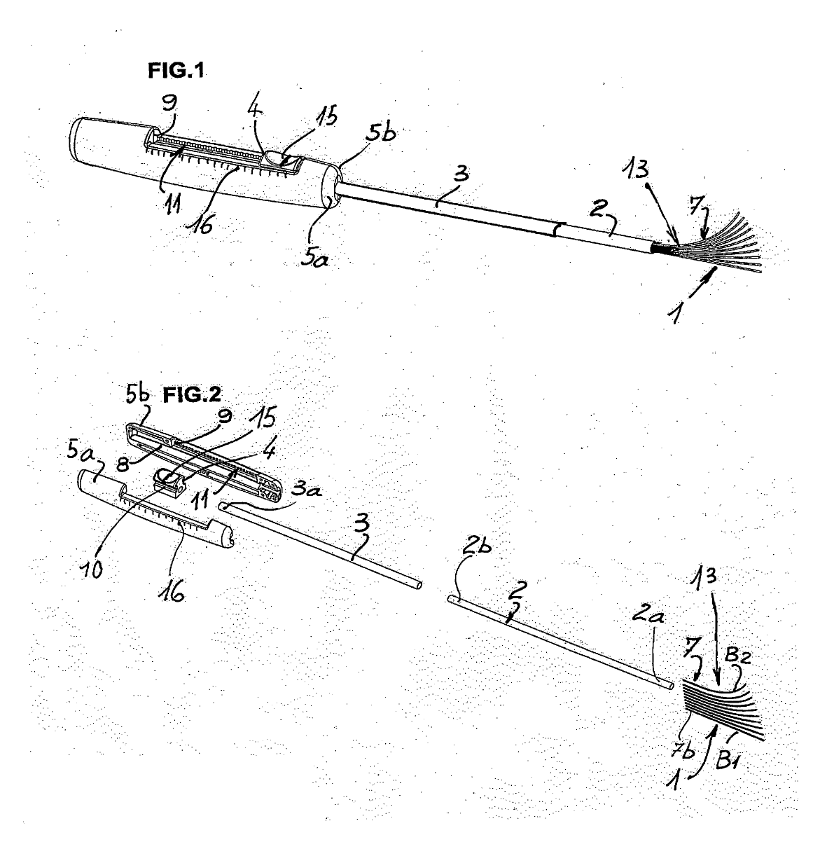

In the embodiment of FIGS. 1 to 5, the reference numeral 1 designates the scraping member, the reference numeral 2 designates a longitudinal body, the reference numeral 3 designates a tubular body in which the first one is slidable, the reference numeral 4 designates a slide displaceable in a two-part handle 5a-5b, and the reference numeral 7 designates the strands forming the brush.

The tubular body 3 is linked by its posterior end to the handle 5a-5b. It has a circular section and extends over a length in the order of 280 millimeters, so that when its anterior end 3a is near the scraping area, the handle is out of the thorax for all the morphologies that can be encountered.

It is made of a rigid material, such as a metal or a sterilizable synthetic material, and is compatible with the material constituting the longitudinal body 2, such as metal or sterilizable plastic material, so that the latter can slide without seizing.

The longitudinal body 2 has a cylindrical section allowing it to slide in the tubular body 3 with reduced radial clearance. The anterior end 2a of the body 2 is linked, by bonding, welding or brazing, to the posterior ends 7b of the strands 7, made of metal. By its posterior end 2b, the longitudinal body 2 is secured to the slide 4 slidably mounted in guide rails 8 of the two-part handle 5a-5b.

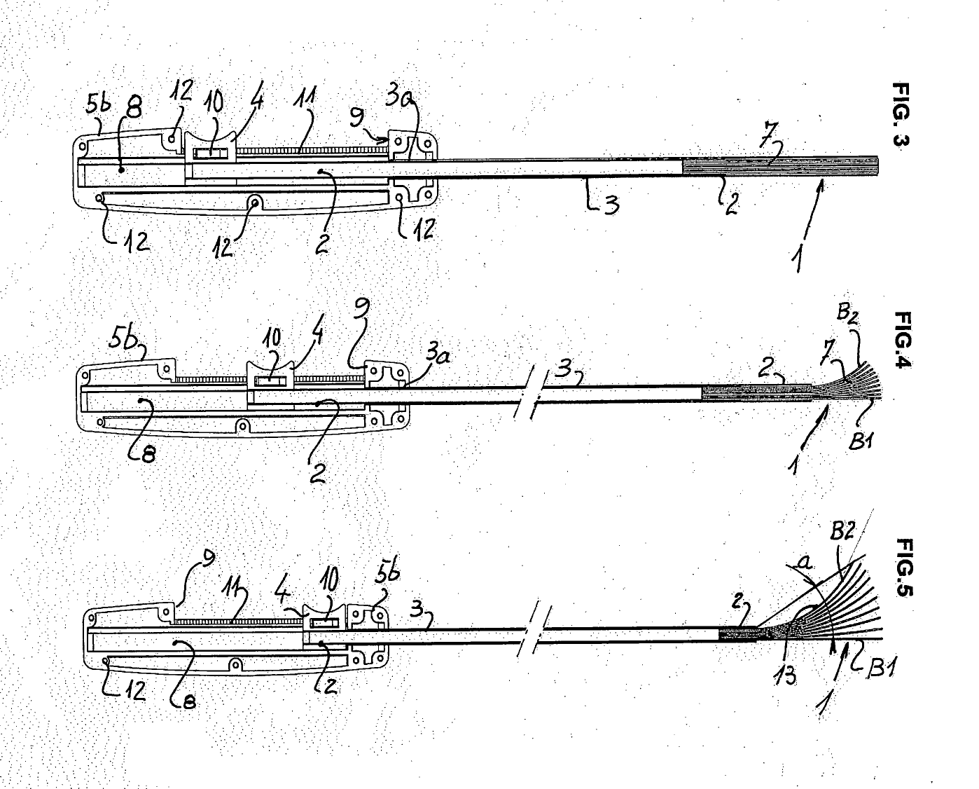

The slide 4 passes through a lumen 9 formed in the handle and thus forms a slider for adjusting the position of the strands 7 between a retracted storage position in the tubular body 3, as shown in FIG. 3, and a fully extended working position, shown in FIG. 5.

The slide 4 also carries elastic tongues 10 cooperating with notches 11 formed on the edges facing the lumen 9.

Tongues 10 and notches 11 can also form indexing means, but in a variant the indexing function is ensured by a transverse index 15 carried by the slide 4 and which can be displaced facing graduations 16 formed on the edges of the lumen 9. The means 15 and 16 are very visible by the operator and complete the tactile sensation he perceives by the engagement of the tongues 10 in the notches 11.

Thus, at any time, the surgeon knows the position of the brush relative to the handle and relative to the engagement of this tool in the body.

By acting on the length of the strands coming out of the tubular body 3, the operator can, as shown in FIG. 4 for a half-stroke coming out, obtain a stiffness different from that obtained by a complete coming out of the strands, shown in FIG. 5, and can therefore adapt this stiffness to the needs of scraping of the parietal pleura.

The two portions 5a-5b of the handle are obtained by molding or machining plastic material, or any other rigid material, and are assembled in the longitudinal median plane of the handle. They are linked to one another by transverse screws screwing into the wells 12, shown in FIGS. 3 to 5. In a variant, the two half-shells are linked by bonding, brazing or welding.

In the shown embodiment, the strands 7 of the brush are provided with cambers 13 having different radii of curvature so as to form, when they are juxtaposed in the working position, as shown in FIGS. 4 and 5, a fan-shaped brush whose strands are distributed on a plane. They extend between a strand B1, disposed parallel to the longitudinal axis of the brush, and a laterally projecting strand B2. In this embodiment and in the working position, the angle a has a value in the order of 30 degrees, as shown in FIG. 5.

In the storage position, shown in FIG. 3, all the strands 7 are disposed parallel to each other, by forming a bundle inside the end of the tubular body 3, then constituting their case.

FIGS. 4 and 5 show that the more the length of the coming out strands is great, the more the angulation of the formed flat fan is enlarged and, the more their flexibility increases, and therefore the more their stiffness decreases. It also shows that the more the strands are coming out, the more they spread apart laterally by increasing the spatial extent of the scraping.

In an embodiment, the strands are made of stainless steel wires having a diameter comprised between 0.15 and 0.40 mm.

The surgeon can thus, during his operation of abrasion of the pleura, modify the extent of the obtained fan, in order to adapt it to the surface to be scraped, as well as the stiffness of the brush, by simple manual displacement of the slide 4 relative to the handle. This is here an important advantage that cannot be carried out with the current means.

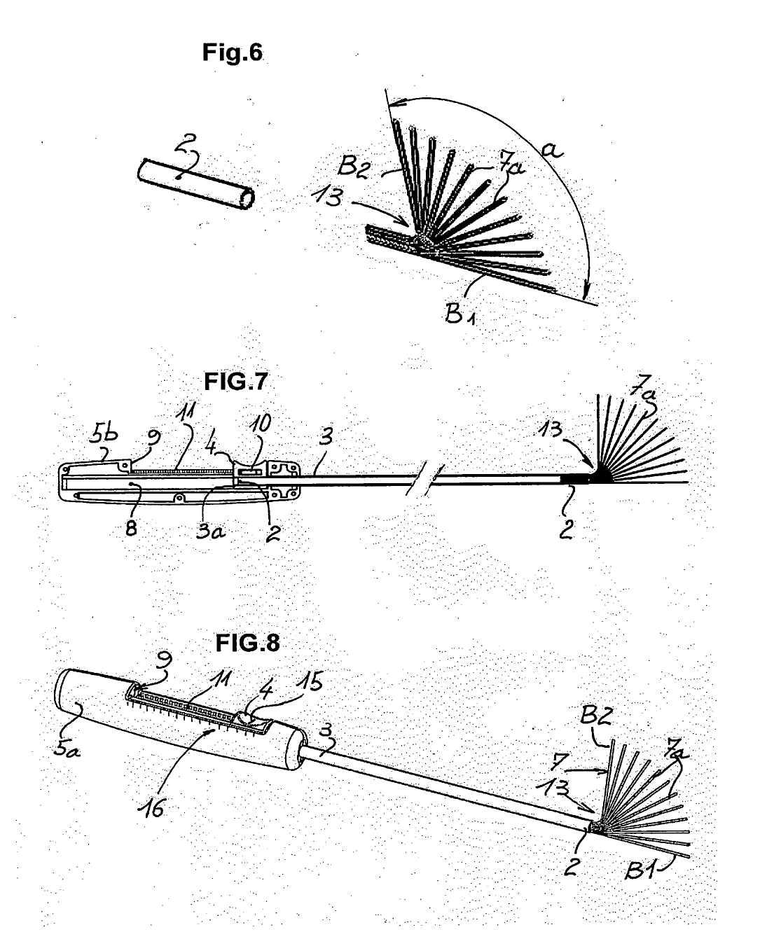

In the variant shown in FIGS. 6 to 8, in which the components have the same reference as before, the strands 7 form a fan extending over a greater angle a in the order of 90 degrees between the strands B1 and B2.

When these strands are in the working position, as shown in FIGS. 7 and 8, the ends of the strands 7 can provide effective scraping throughout the periphery of the fan, both by the end strands B1 or B2 and the intermediate strands.

The greatest angular opening of the fan does not interfere in any way with the operation of retraction of the strands in the tubular body 2 since, by the elasticity of their cambers 13, these strands fold up longitudinally one against the other.

With this arrangement, the surgeon can give the brush the configurations of FIG. 4, 5 or 7, being differentiated by the stiffness of the strands and by the angular distribution of the scraping, and this in full knowledge of these inner parameters, thanks to the indexation of the body 2 in the handle 5a-5b.

1. A brush for the abrasion of the parietal pleura, the brush comprising:
a longitudinal body carrying at one of its ends a plurality of strands forming the scraping member,
a tubular body surrounding the longitudinal body,
a handle secured to the tubular body and containing longitudinal displacement means of the other body, between at least one working position, in which the working member protrudes out of the free end of the tubular body, and one storage position, in which this member is completely retracted into the tubular body,
wherein the means for displacing the longitudinal body carrying the strands, are constituted by a manually-actuated slide linked to the posterior end of the longitudinal body, which is opposite to that carrying the strands, said slide being movable in a longitudinal lumen of the handle and cooperating with indexing means carried by the edges of this lumen, whereas the strands include elastically deformable cambers and having different radii of curvature, so as to form, when they are retracted in the tubular body, a bundle of parallel strands and, when they are in the working position, out of the tubular body, a flat fan-shaped brush, whose strands extend laterally from the longitudinal axis.

2. The brush for the abrasion of the parietal pleura according to claim 1, wherein the handle contains inner guide rails on which the slide can move.

3. The brush for the abrasion of the parietal pleura according to claim 1, wherein the angle a of the fan formed by the strands, measured between, on the one hand, a strand B1 parallel to the longitudinal axis of the body carrying the strands and the most cambered strand B2 on the side, has a value which decreases from 180 degrees to 90 degrees as the strands move away from the strand B1 parallel to the longitudinal axis.

4. The brush for the abrasion of the parietal pleura according to claim 1, wherein the handle is composed of two rigid material portions assembled in the longitudinal median plane, whereas the tubular body is made of metal or plastic material and in that the longitudinal body is made of a metal or plastic material which promotes its displacement in the tubular body forming a protective case.

5. The brush for the abrasion of the parietal pleura according to claim 1, wherein the edges of the longitudinal lumen are provided with a plurality of notches cooperating with at least one elastic tongue carried by the slider.

6. The brush for the abrasion of the parietal pleura according to claim 1 taken together, wherein the slider carries a transverse index cooperating with a graduation formed on the edges of the lumen.
